(19) United States
(12) Patent Application Publication
(10) Pub. No.: US 2009/0317496 A1
(43) Pub. Date: Dec. 24, 2009

(54) Method for Preventing and Treating the Disease Caused by Vascular Damage and the Use Thereof

(75) Inventors: Jeong Hill Park, Seoul (KR); Hyun Young Kim, Gyeonggi-do (KR); Young Guen Kwon, Seoul (KR); Jeong Hun Kim, Seoul (KR); Bok Deuk Kim, Seoul (KR); Young Sun Maeng, Seoul (KR)

Correspondence Address: KIRK HAHN, 14431 HOLT AVE, SANTA ANA, CA, 92705, US

(73) Assignee: GINSENG SCIENCE INC., Seoul (KR)

(21) Appl. No.: 12/067,044

(22) PCT Filed: Sep. 15, 2006
(86) PCT No.: PCT/KR2006/003699

§ 371 Date: Mar. 14, 2008

(30) Foreign Application Priority Data
Sep. 16, 2005 (KR) 10-2005-0086650

(51) Int. Cl. A61K 36/258; A61P 3/10; C07H 15/00; A61K 31/704

(52) U.S. Cl. 424/728; 536/5; 514/26

(57) ABSTRACT

The present invention relates to compositions containing an extract of processed Panax genus plant and saponin compounds isolated therefrom for treat the diseases caused by vascular injury in human or mammal, the method for treating said diseases using thereby and the use thereof. The extract of processed Panax genus plant and saponin compounds isolated therefrom shows suppressive effect on endothelial dysfunction and damage of blood-retinal on HUVEC and retinal vascular endothelial cell. Therefore, it can be used as the therapeutics or functional health food for treating and preventing the diseases caused by vascular injury.

### BACKGROUND OF THE INVENTION

1. Technical Field

The present invention relates to compositions containing an extract of processed Panax genus plant and saponin compounds isolated therefrom for treat the diseases caused by vascular damage in human or mammal, the method for treating said diseases using thereby and the use thereof. More particularly, the present invention relates to a use of an extract of processed ginseng and saponin compounds isolated therefrom with enhanced pharmacological effects due to heat-treatment of a Panax genus plants so as to make a ratio of ginsenoside (Rg3+Rg5+Rk1) to (Rb1+Rb2+Rc+Rd) of over 1.0.

2. Background Art

Blood vessels provide oxygen and adequate nutrients to the cells of all organs as a pathway of blood delivery and plays important roles in maintaining cell homeostasis by eliminating wastes matter from the system as a main organ. Arterial wall consists of three layers, i.e., tunica intima, tunica media and tunica adventitia. Tunica intima consists of endothelial cell and elastic fiber covering therewith and tunica media consists of ring-shaped smooth muscles and elastic fiber. Tunica adventitia consists of mainly connective-tissue linking vein with neighboring tissue.

Blood endothelial cell layer plays a role in secreting various physiologically active substances, and controlling blood extension, blockage of thrombosis, the permeation and transfer of selective metabolites in vessel wall, and blood flow and adhering white blood cell and thrombocytic cell with the cell surface. Angioblast, a precursor of endothelium produced from mesoderm during development stage is differentiated into various blood endothelial cells in various organs of human body. Those organ-specific endothelia are presumed to be caused by surrounding cells or environmental factors in organ (Jain R K., Nat. Med., 9, pp. 685-693, 2003).

Various factors occurring in blood endothelial cell layer, for examples, physical factors such as pulsatile stress on blood cell wall or the tension on blood vessel; various humoral factors such as excessively abundant lipid and glucose in blood; and other factors such as the depletion of oxygen and nutrients caused by blood occlusion or active oxygen etc, causes to the cell death of blood endothelial cell, abnormality of tight junction complex and early-aging etc. There have been reported that the abnormality of blood vessel function results in the main causes of various human diseases such as vasculitis symptoms, myocardial infarction, ischemic brain injury, diabetic retinitis etc. Therefore, the substance preventing from the death and the damage of endothelial cells such as the depletion of tight-junction and enhancing the homeostasis of blood cell function may be used usefully in prevention and treatment of various vascular diseases (O'Riordan E et al., Kidney Int., 67(5), pp. 1654-1658, 2005; Oda M et al., Clin Hemorheol Microcir., 23, pp. 199-211, 2000; Brandes R P et al., Cardiovasc. Res., 66(2), pp. 286-294, 2005). Especially, there has been reported that both of retinopathy of prematurity in infant and diabetic retinopathy in adult, representative severe diseases in eye occur by the mechanism as follows: retina blood barrier is destroyed by the action of various factors such as VEGF released from ischemic premature retina blood and the injury of angio-endothelial cell in case of retinopathy of prematurity (Gariano R F et al., Surv. Opthalmol., 40 pp 481-90, 1996); and the permeation of retinal blood is increase resulting from the destruction of retinal blood barrier retina and retinal ischemia occurs due to the injury of angio-endothelial cell in case of diabetic retinopathy (Cogan D G et al., Arch. Ophthalmology, 66, pp 366-78, 1961; Palmer E A et al., Ophthalmology 98, pp 1620-4-, 1991). Therefore, there have been needed to develop new agent to inhibit the destruction of retinal blood barrier and prevent the injury of angio-endothelial cell till now.

It is known that there are many genus of Panax genus plants belonged to Araliaceae, for example, Panax ginseng distributed or cultivated in far-eastern Asia region, Panax quinquefolia in America and Canada, Panax notoginseng in China, Panax trifolia in eastern region of north America, Panax japonica in Japan, China and Nepal, Panax pseudoginseng in Nepal, Panax vietnamensis in Vietnam, Panax elegatior, Panax wangianus and Panax bipinratifidus etc.

It has been known that the main constituent of Panax genus plant is dammarane-skeleton type saponins, such as ginsenosides Rb₁, Rb₂, Rc, Rd, Rg1 and Re etc. The biological activities of them are different from each other in accordance with their chemical structures and ginseng has been reported to be effective in fatigue recovery or vigor reinforcement from long years ago.

Recently, there have been several attempts to strengthen pharmacological effects of ginseng by modifying the method of ginseng processing for example, Park et al developed new methods for preparing a processed ginseng under specific high temperature and high pressure as disclosed in Korean Patent Registration No. 192678 and U.S. Pat. No. 5,776,460, which changes main ginseng saponins such as ginsenosides Rb1, Rb2, Rc and Rd, into new saponin metabolites such as ginsenosides Rg3, Rg5, and Rk1 showing new and more potent pharmacological effects, for examples, anti-oxidative activity, anti-cancer activity and alleviating activity of blood circulation etc (Kim W Y et al., J. Nat. Prod., 63(12), pp. 1702-1704). Especially, the pharmacological effect of ginsenosides Rg3, Rg5 and Rk1 has known to be strongest among them and those ginsenosides produced through said new processing method are formed by the process that a part of sugar moiety in dammarane glycoside, i.e. ginsenosides Rb1, Rb2, Rc and Rd, was cleaved and continuously subjected to dehydration reaction at the position of C-20 (See FIG. 1). Accordingly, these new metabolites can be produced in the root, stem or leaf of any panax genus plants such as Panax ginseng, Panax quinquefolia, Panax notoginseng, Panax trifolia, Panax japonica, Panax pseudoginseng, Panax vietnamensis, Panax elegatior, Panax wangianus and Panax bipinratifidus which contains dammarane glycoside through the processing method of Park et al (Korean Patent Registration No. 192678 and U.S. Pat. No. 5,776,460).

However, there has been not reported or disclosed about the treating or preventing effect of the processed ginseng extract prepared by above-described methods on the disease caused by vascular damage in any of above cited literatures, the disclosures of which are incorporated herein by reference.

Accordingly, the present inventors have discovered that the processed ginseng product as to make a ratio of ginsenoside (Rg3+Rg5+Rk1) to (Rb1+Rb2+Rc+Rd) of over 1.0 and the extract thereof show potent suppressive effect on endothelial dysfunction and the damage of blood-retinal barrier and finally completed the present invention.

### SUMMARY OF THE INVENTION

The present invention also provides a use of extract of processed ginseng and saponin compounds isolated therefrom for the prevention or treatment of the diseases caused by vascular injury.

The present invention provides a pharmaceutical composition and a health food comprising extract of processed ginseng and saponin compounds isolated therefrom as an active ingredient in an effective amount to treat and prevent the diseases caused by vascular injury.

The present invention also provides a method for treating or preventing of the diseases caused by vascular injury in human or mammal comprising administrating to said mammal an effective amount of above extract of processed ginseng and saponin compounds isolated therefrom, together with a pharmaceutically acceptable carrier thereof.

### DISCLOSURE OF THE INVENTION

In accordance with the present invention, the present invention provides a use of extract of processed ginseng and the saponin compounds isolated therefrom for the preparation of medicament employed for preventing or treating the diseases caused by vascular injury in human and mammals.

In accordance with the present invention, the present invention also provides a composition comprising an extract of processed ginseng and saponin compounds isolated therefrom as an active ingredient in an effective amount to treat and prevent the diseases caused by vascular injury.

The present invention also provides a method for treating or preventing of the diseases caused by vascular injury in human or mammal comprising administrating to said mammal an effective amount of an extract of processed ginseng and saponin compounds isolated therefrom, together with a pharmaceutically acceptable carrier thereof.

The term “an extract of processed ginseng” disclosed herein comprises the extract of Panax genus plants so as to make a ratio of ginsenoside (Rg3+Rg5+Rk1) to (Rb1+Rb2+Rc+Rd) of over 1.0 as an active ingredient in an effective amount to treat and prevent the diseases caused by vascular damage and an extract of processed ginseng obtained by the steps; treating Panax genus plants with heat at the temperature ranging from 70 to 150° C. for the period ranging from 2 to 6, as an active ingredient in an amount effective to treat or prevent the diseases caused by vascular damage, which can be prepared by the procedure disclosed in Korean Patent Registration No. 192678 and U.S. Pat. No. 5,776,460).

The term ginsenoside “Rg3” disclosed herein includes two isomers of ginsenoside (20-S) and (20-R).

The term “saponin compounds isolated from the extract of processed ginseng” disclosed herein comprise at least one saponin or their combination selected from the group consisting of ginsenoside Rg3, Rg5 and Rk1.

The term “Panax genus” disclosed herein comprises the root, stem, petal, leaf or fruit of Panax ginseng, Panax quinquefolia, Panax notoginseng, Panax trifolia, Panax japonica, Panax pseudoginseng, Panax vietnamensis, Panax elegatior, Panax wangianus or Panax bipinratifidus.

The term “diseases caused by vascular injury” disclosed herein comprises ischemic diseases or diabetic complication.

The term “ischemic diseases” disclosed herein comprises arteriosclerosis, embolism, aging, ischemic heart disease, brain stroke, anigma, cerebral infarction, intracranial hemorrhage, nephrosclerosis, retinopathy of prematurity or myocardial infarction.

The term “intracranial hemorrhage” disclosed herein comprises spontaneous intracranial hemorrhage or subdural hemorrhage.

The term “aneurysm” disclosed herein comprise abdominal aortic aneurysm.

The term “diabetic complication” disclosed herein comprises diabetic retinopathy (visual impairment, retinal hemorrhage), diabetic nephropathy or diabetic peripheral neuropathy. Above-described extract of processed ginseng and saponin compounds isolated therefrom can be prepared in accordance with the following preferred embodiment.

Hereinafter, the present invention is described in detail.

For the present invention, for example, dried plant material of Panax genus, for examples, the root of Panax ginseng is cut into small pieces and the piece was heated at the temperature ranging from 70 to 150° C., preferably from 100 to 130° C., for the period ranging from 2 to 6, preferably 3 to 5 hrs; and was mixed with 1 to 20-fold, preferably, 3 to 10-fold weight of water, C₁-C₄ lower alcohol such as methanol, ethanol, butanol, or the mixtures thereof, preferably ethanol; and was heated for the period ranging from 3 to 10 hours, preferably 3 to 6 hours, by reflux extraction with water, cold water extraction, ultra-sonication or conventional extraction, preferably by reflux extraction with water; the residue was filtered and then the filtrate was dried at the temperature ranging from 40 to 80, preferably from 50 to 70, to obtain inventive extract of processed ginseng.

To obtain purposed ginsenosides from the inventive extract of processed ginseng, following procedure could be performed:

The inventive extract of processed ginseng prepared by above step, is mixed with water, and then is extracted with 1 to 100-fold, preferably, 1 to 5-fold volume of non-polar organic solvent such as hexane, ether, dichloromethane, chloroform, ethyl acetate or the mixtures thereof; the water-soluble layer obtained the above step is further extracted with polar organic solvent such as butanol; and the above organic solvent-soluble extract is further subjected to chromatography for increasing the content of ginsenoside by repeating the above extraction steps. The purposed ginsenosides of the present invention, i.e., ginsenoside Rg3, Rg5 or Rk1 can be obtained by repeated crystallization steps with appropriate solvent such as water, lower alcohol, lower ketone, chloroform or the mixture thereof.

Through the above described procedure, the saponins such as ginsenoside Rb₁, Rb₂, Rc, Rd etc being contained in plant material is transformed into chemically modified ginsenosides such as ginsenoside Rg₃, Rg₅, Rk₁ etc due to heat treatment.

In particular, the extract of processed ginseng according to the present invention wherein a ratio of ginsenoside (Rg3+Rg5+Rk1) to (Rb1+Rb1+Rc+Rd) of over 1.0 showing more potent physiological activities than those of extract of non-processed ginseng.

In order to perform purposed use of the present invention, the use of the present invention may be embodied by following inventive composition. The inventive composition may additionally comprise conventional carrier, adjuvants or diluents in accordance with a using method. It is preferable that said carrier is used as appropriate substance according to the usage and application method, but it is not limited. Appropriate diluents are listed in the written text of Remington's Pharmaceutical Science (Mack Publishing co, Easton Pa.).

Hereinafter, the following formulation methods and excipients are merely exemplary and in no way limit the invention.

The composition according to the present invention can be provided as a pharmaceutical composition containing pharmaceutically acceptable carriers, adjuvants or diluents, e.g., lactose, dextrose, sucrose, sorbitol, mannitol, xylitol, erythritol, maltitol, starches, acacia rubber, alginate, gelatin, calcium phosphate, calcium silicate, cellulose, methyl cellulose, polyvinyl pyrrolidone, water, methylhydroxy benzoate, propylhydroxy benzoate, talc, magnesium stearate and mineral oil. The formulations may additionally include fillers, anti-agglutinating agents, lubricating agents, wetting agents, flavoring agents, emulsifiers, preservatives and the like. The compositions of the invention may be formulated so as to provide quick, sustained or delayed release of the active ingredient after their administration to a patient by employing any of the procedures well known in the art.

For example, the compositions of the present invention can be dissolved in oils, propylene glycol or other solvents that are commonly used to produce an injection. Suitable examples of the carriers include physiological saline, polyethylene glycol, ethanol, vegetable oils, isopropyl myristate, etc., but are not limited to them. For topical administration, the compounds of the present invention can be formulated in the form of ointments and creams.

Pharmaceutical formulations containing present composition may be prepared in any form, such as oral dosage form (powder, tablet, capsule, soft capsule, aqueous medicine, syrup, elixirs pill, powder, sachet, granule), or topical preparation (cream, ointment, lotion, gel, balm, patch, paste, spray solution, aerosol and the like), or injectable preparation (solution, suspension, emulsion).

The composition of the present invention in pharmaceutical dosage forms may be used in the form of their pharmaceutically acceptable salts, and also may be used alone or in appropriate association, as well as in combination with other pharmaceutically active compounds.

The desirable dose of the inventive extract or composition varies depending on the condition and the weight of the subject, severity, drug form, route and period of administration, and may be chosen by those skilled in the art. However, in order to obtain desirable effects, it is generally recommended to administer at the amount ranging 0.01-10 g/kg, preferably, 1 to 5 g/kg by weight/day of the inventive extract or compounds of the present invention. The dose may be administered in single or divided into several times per day. In terms of composition, the complex herbal composition should be present between 0.01 to 80% by weight, preferably 0.5 to 50% by weight based on the total weight of the composition.

The pharmaceutical composition of present invention can be administered to a subject animal such as mammals (rat, mouse, domestic animals or human) via various routes. All modes of administration are contemplated, for example, administration can be made orally, rectally or by intravenous, intramuscular, subcutaneous, intracutaneous, intrathecal, epidural or intracerebroventricular injection.

The present inventors demonstrated that the inventive extract of the present invention showed more potent activity of the disease caused by vascular injury than that of the extract of processed ginseng prepared by conventional method or simple processed method, by accomplishing in vitro and in vivo experiment, e.g., suppressive effect on endothelial dysfunction and blood-retinal barrier permeation etc., therefore, it has been confirmed that above described inventive composition is very useful in the prevention or treatment of the disease caused by vascular injury.

In particular, the composition of the present invention also potent preventing activity of the diseases caused by vascular injury, thus it is very useful for patients susceptible with the various diseases caused by vascular injury.

Accordingly, it is another object of the present invention to provide a health care food comprising above described extract or saponin compounds isolated therefrom prepared by above processes and a sitologically acceptable additive to prevent or alleviate the diseases caused by vascular injury.

The present invention also provides a health care food comprising the extract of processed ginseng obtained by the steps; heating Panax plant with heat at the temperature ranging from 70 to 150° C. for the period ranging from 2 to 6, and a sitologically acceptable additive to prevent or alleviate the diseases caused by vascular injury.

The present invention also provides a health care food comprising saponin compounds at least one or their combination selected from the group consisting of ginsenoside Rb3, Rg5 and Rk1 and a sitologically acceptable additive to prevent or alleviate the diseases caused by vascular injury.

Above-described composition therein can be added to food, additive or beverage for prevention of the diseases caused by vascular injury. For the purpose of preventing the diseases caused by vascular injury, wherein, the amount of above described extract or compound in food or beverage may generally range from about 0.1 to 15 w/w %, preferably 1 to 10 w/w % of total weight of food for the health food composition and 1 to 30 g, preferably 3 to 10 g on the ratio of 100 m [symbol] of the health beverage composition.

Providing that the health beverage composition of present invention contains above described extract or compounds as an essential component in the indicated ratio, there is no particular limitation on the other liquid component, wherein the other component can be various deodorant or natural carbohydrate etc such as conventional beverage. Examples of aforementioned natural carbohydrate are monosaccharide such as glucose, fructose etc; disaccharide such as maltose, sucrose etc; conventional sugar such as dextrin, cyclodextrin; and sugar alcohol such as xylitol, and erythritol etc. As the other deodorant than aforementioned ones, natural deodorant such as taumatin, stevia extract such as levaudioside A, glycyrrhizin et al., and synthetic deodorant such as saccharin, aspartam et al., may be useful favorably. The amount of above described natural carbohydrate is generally ranges from about 1 to 20 g, preferably 5 to 12 g in the ratio of 100 m [symbol] of present beverage composition.

The other components than aforementioned composition are various nutrients, a vitamin, a mineral or an electrolyte, synthetic flavoring agent, a coloring agent and improving agent in case of cheese chocolate et al., pectic acid and the salt thereof, alginic acid and the salt thereof, organic acid, protective colloidal adhesive, pH controlling agent, stabilizer, a preservative, glycerin, alcohol, carbonizing agent used in carbonate beverage et al. The other component than aforementioned ones may be fruit juice for preparing natural fruit juice, fruit juice beverage and vegetable beverage, wherein the component can be used independently or in combination. The ratio of the components is not so important but is generally range from about 0 to 20 w/w % per 100 w/w % present composition.

Examples of addable food comprising aforementioned extract or compounds therein are various food, beverage, gum, vitamin complex, health improving food and the like.

Inventive extract or compound of the present invention has no toxicity and adverse effect therefore they can be used with safe.

The present invention is more specifically explained by the following examples. However, it should be understood that the present invention is not limited to these examples in any manner.

### BRIEF DESCRIPTION OF DRAWINGS

The above and other objects, features and other advantages of the present invention will more clearly understood from the following detailed description taken in conjunction with the accompanying drawings, in which;

FIG. 1 shows the structural change of ginsenoside due to heat treatment of Panax plant,

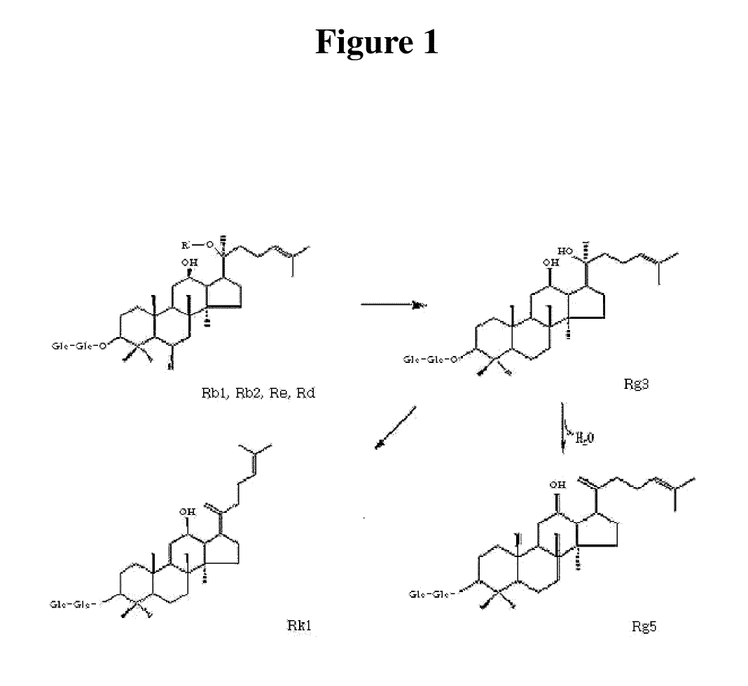

FIG. 2 shows the cultured cell of HUVEC and retinal vascular endothelial cell,

FIG. 3 is the result of MTT assay showing suppressive effect of SG on the apoptosis of HUVEC (a); of SG (b) and of ginsenosides Rb1, Rg1, Re, Rg3, Rg5, Rk1 on retinal vascular endothelial cell (c),

FIG. 4 is the result of MTT assay showing suppressive effect of Rk1 on the apoptosis of HUVEC in medium,

FIG. 5 is the result of staining with DAPI showing suppressive effect of Rk1 on the apoptosis of HUVEC in medium,

FIG. 6 is the result of western blot analysis showing recovery effect of Rk1 on the decrease of tight junction protein induced by vascular endothelial growth factor and by AGE on human retinal endothelial cell (a) and recovery effect of Rk1 on the tight junction protein of mouse retina (b),

FIG. 7 is the result of immuno-cytochemical staining method showing recovery effect of Rk1 on the decrease of tight junction protein caused by AGE on human retina endothelial cell,

FIG. 8 is the result of immuno-cytochemical staining method showing recovery effect of Rk1 on the decrease of tight junction protein caused by vascular endothelial growth factor on human retinal endothelial cell,

FIG. 9 is the result of immuno-cytochemical staining method showing recovery effect of Rk1 on the decrease of tight junction protein caused by vascular endothelial growth factor on mouse retina,

FIG. 10 is the result of immuno-cytochemical staining method showing recovery effect of Rk1 on the decrease of tight junction protein caused by diabetes on mouse retina,

FIG. 11 is the result of angiography analysis showing recovery effect of Rk1 on the permeability increase of blood vessel in retina caused by vascular endothelial growth factor and diabetes on mouse retina.

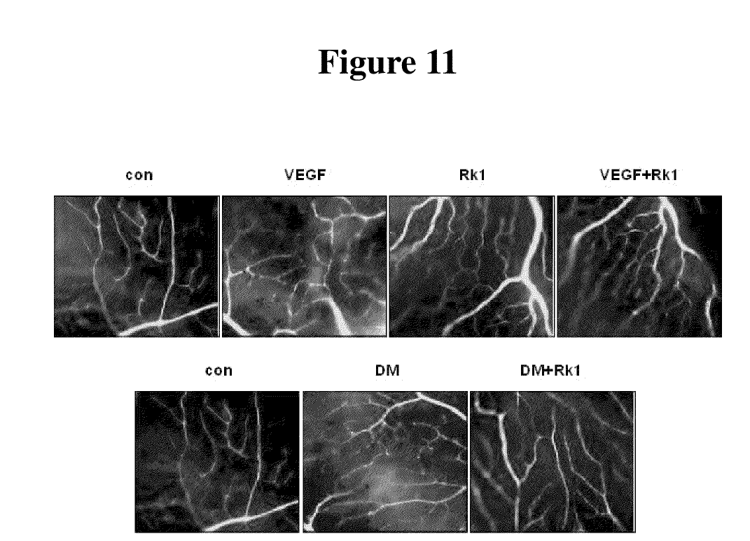

### BEST MODE FOR CARRYING OUT THE INVENTION

It will be apparent to those skilled in the art that various modifications and variations can be made in the compositions, use and preparations of the present invention without departing from the spirit or scope of the invention.

The present invention is more specifically explained by the following examples. However, it should be understood that the present invention is not limited to these examples in any manner.

### EXAMPLES

The following Reference Example, Examples and Experimental Examples are intended to further illustrate the present invention without limiting its scope.

### Example 1

### Preparation of the Extract of Processed Ginseng

Specifically processed ginseng was prepared in accordance with the procedure disclosed in the literature (Kim W Y et al., J. Nat. Prod., 63(12), pp. 1702-1704; Kwon S W et al., J. Chromatogr A., 921(2), pp. 335-339, 2001).

1 Kg of dried plant material of Panax genus, for examples, the root of Panax ginseng was cut into small pieces and the sliced piece was heated at 120° C. for 4 hours in autoclave. The processed ginseng was mixed with 2 [symbol] of ethanol and heated for 4 hours by reflux extraction with water. The residue was filtered and then the filtrate was evaporated to obtain inventive extract of processed ginseng, which designated as ‘SG.’ hereinafter.

### Example 2

### Preparation of Ginsenoside Rg3, Rg5 and Rk1 Isolated from the Extract of Processed Ginseng

10 g of processed ginseng extract prepared by the above Example 1 was mixed with 100 m [symbol] of water, and then was extracted with 100 m [symbol] of ether 3 times. The remaining water-soluble layer was further extracted with 100 m [symbol] of butanol 3 times. The butanol soluble extract was further subjected to silica gel column chromatography and eluted with a ethylacetate:methanol:water mixture (20:1:1) to obtain 500 mg of the fraction containing ginsenoside Rg3 by repeating the above chromatography procedure. The fraction containing ginsenosides Rg5 and Rk1 was further purified over semi-preparative HPLC using reverse phase column with 60% CH₃CN eluent. 200 mg of Rg5 and 150 mg of Rk1 were obtained.

### Experimental Example 1

### Suppression Effect on Vascular Endothelial Cell Injury

### 1-1. Vascular Endothelial Cell Culture

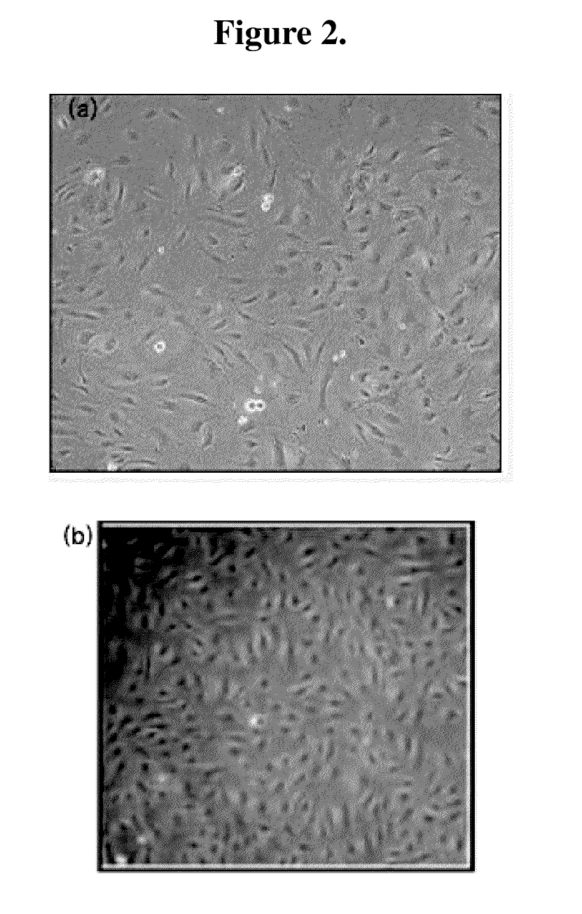

The HUVEC (human umbilical venous endothelial cell) and retinal vascular endothelial cell (cell-systems, USA) were incubated at M199 medium containing 20% (w/v) FBS (HyClone, Canada), 100 units/m [symbol] of penicillin (Invitrogen, USA), 100 μg/m [symbol] of streptomycin (Invitrogen, USA), 3 ng/m [symbol] of bFGF (basic fibroblast growth factor, Upstate Biotechnology, USA) and 5 units/m [symbol] of heparin (Life Technologies, USA) under 5% CO₂ gas condition at 37° C. (See FIG. 2a and FIG. 2b).

### 1-2. Suppression Effect of SG on Retinal Vascular Endothelial Cell Apoptosis Caused by the Lack of Serum

To investigate the suppression effect of SG was determined by (3-[4,5-dimethylthiazol-2-yl]-2,5-diphenyl tetrazolium bromide (MTT) assay method disclosed in the literature (Wang Z et al., Biol., Pharm. Bull., 24, pp 159-162, 2001).

HUVEC and retinal vascular endothelial cell were spread on 24-well plates (3×10⁴ cells/well) and were incubated for 24 hours. The mediums were substituted with new medium containing 20% (w/v) FBS and FBS free medium respectively. The FBS free medium was treated with various concentrations of SG, i.e., 0, 1, 5, 10, 20 μg/m [symbol] . The medium was incubated for 24 hours. After the incubation, MTT assay was performed.

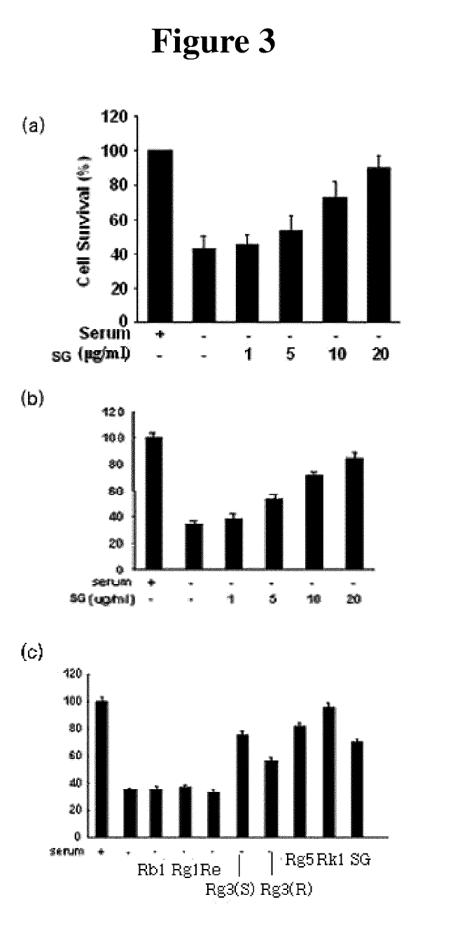

As shown in FIG. 3a and FIG. 3b, SG inhibited the apoptosis of HUVEC and retinal vascular endothelial cells and increased the cell viability of HUVEC and retinal vascular endothelial cells in a dose dependent manner.

### 1-3. Suppressive Effect of SG, Rb1, Rg1, Re, Rg3, Rg5 and Rk1 on the Cell Apoptosis if Retinal Vascular Endothelial Cell Caused by the Lack of Serum

To investigate the suppressive effect of SG, Rb1, Rg1, Re, Rg3, Rg5 and Rk1 on the cell apoptosis if retinal vascular endothelial cell caused by the lack of serum, the effect were determined by (3-[4,5-dimethylthiazol-2-yl]-2,5-diphenyl tetrazolium bromide (MTT) assay method disclosed in the literature (Wang Z et al., Biol., Pharm. Bull., 24, pp 159-162, 2001).

HUVEC and retinal vascular endothelial cell were spread on 24-well plates (3×10⁴ cells/well) and were incubated for 24 hours respectively. The mediums were substituted with new medium containing 20% (w/v) FBS and FBS free medium respectively. The FBS free medium was treated with 10 μg/m [symbol] of SG, Rb1, Rg1, Re, Rg3, Rg5 and Rk1 and the medium was incubated for 24 hours. After the incubation, MTT assay was performed.

As shown in FIG. 3c, SG, Rg3, Rg5 and Rk1 inhibited the apoptosis of HUVEC and retinal vascular endothelial cells and increased the cell viability of HUVEC and retinal vascular endothelial cells in a dose dependent manner while Rb1, Rg1 and Re did not.

### 1-4. Suppressive Effect of Rk1 on the Cell Apoptosis of Vascular Endothelial Cell Caused by the Lack of Serum

Vascular endothelial cell were spread on 24-well plates (3×10⁴ cells/well) and were incubated for 24 hours. The mediums were substituted with new medium containing 20% (w/v) FBS and FBS free medium respectively. The FBS free medium was treated with various concentrations of Rk1, i.e., 0, 0.5, 1, 5, 10 μg/m [symbol] and incubated for 24 hours. After the incubation, MTT assay was performed.

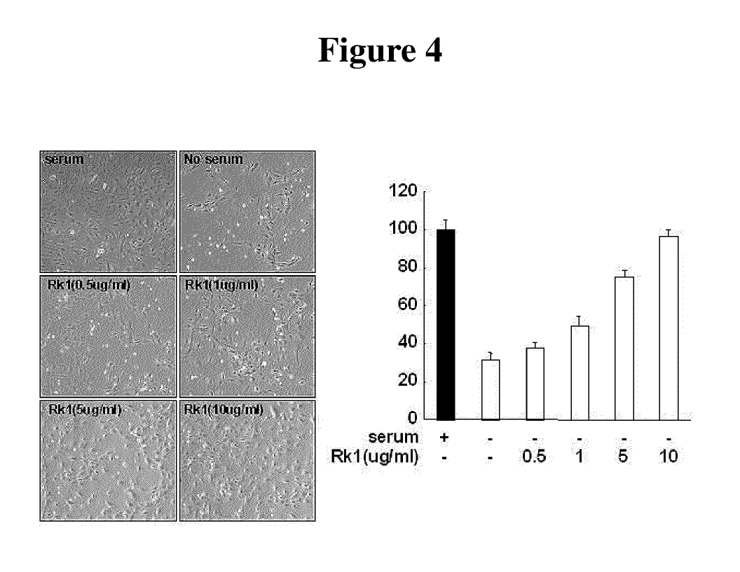

As shown in FIG. 4, Rk1 inhibited the apoptosis of vascular endothelial cells and increased the cell viability of vascular endothelial cells in a dose dependent manner.

### 1-5. Suppressive Effect of Rk1 on the DNA Fragmentation of Vascular Endothelial Cell Caused by the Lack of Serum

Vascular endothelial cell were spread on 35 mm dish (1.5×10⁵ cells/well) and were incubated for 24 hours. The mediums were substituted with new medium containing 20% (w/v) FBS and FBS free medium respectively. The FBS free medium was treated with various concentrations of Rk1, i.e., 0, 0.5, 1, 5, 10 μg/m [symbol] and incubated for 24 hours. After the incubation, the medium was washed with PBS solution 2 times, fixed with 2% of paraformaldehyde and washed with PBS solution 2 times again. DAPI (4′,6-Diamidino-2-phenylindole-2-HCl. Calbiochem, USA) solution was added thereto, incubated for 30 minutes in darkroom and washed with PBS two times. The DNA fragmentation was measured by fluorescent microscope (Fluorescent microscope, Axioplan2, ZEISS, Germany) after mounting with cover slip.

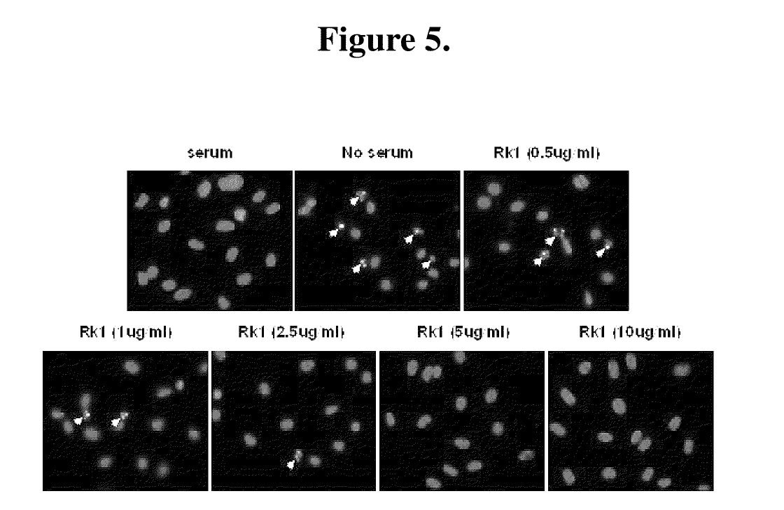

As shown in FIG. 5, ginsenoside Rk1 suppressed the DNA fragmentation of vascular endothelial cells in a dose dependent manner.

### Experimental Example 2

### Suppression Effect on the Destruction of Blood Retinal Barrier

### 2-1. Recovering Effect of Rk1 on the Decrease of Tight Junction Protein Caused by Vascular Endothelial Growth Factor and AGE

Human retinal vascular endothelial cell were spread on 60 mm dish (3×10⁵ cells/well) and were incubated for 24 hours.

The cells were transferred to human endothelial SFM-basal growth medium (Invitrogen, USA), incubated for 24 hours and transferred to new human endothelial SFM-basal growth medium again. The medium was treated with 20 ng/m [symbol] of VEGF (vascular endothelial growth factor) and Rk1, and incubated for 6 hours. After the incubation, the cells were collected to induce lysis and western blot analysis was performed.

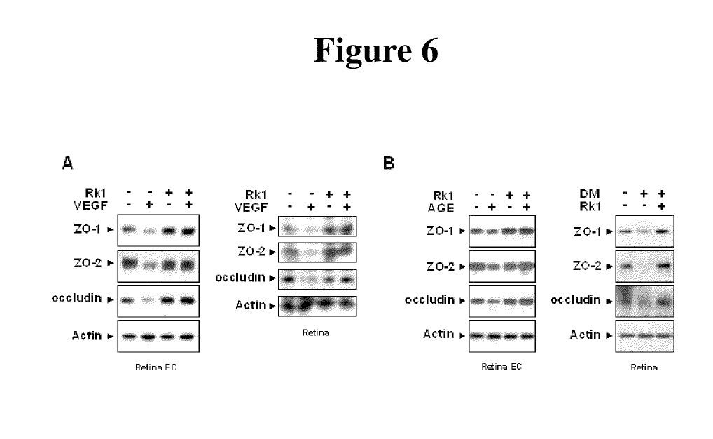

As shown in FIG. 6a, Rk1 show potent recovering effect on the decrease of all tight junction proteins, i.e., ZO-1, ZO-2 and occludin which was caused by VEGF and AGE (advanced glycation end product).

### 2-2. Recovery Effect of Rk1 on the Decrease of Tight Junction Protein Caused by Vascular Endothelial Growth Factor and Diabetes

Both of 100 ng of VEGF and 30 μg of Rk1 were injected into the retina of C57/BL6 mice (Orient, Korea). After 24 hours, the extracted retina from mice was induced to lysis and western blot analysis was performed.

As shown in FIG. 6b, Rk1 showed potent recovering effect on the decrease of all tight junction proteins, i.e., ZO-1, ZO-2 and occludin which was caused by VEGF and diabetes.

### 2-3. Immunocytochemistry Analysis (1)

Human retinal vascular endothelial cell were spread on 35 mm dish (2×10⁵ cells/well) and were incubated for 24 hours. The cells were transferred to human endothelial SFM-basal growth medium (Invitrogen, USA), incubated for 24 hours and transferred to new human endothelial SFM-basal growth medium again. The medium was treated with 20 ng/m [symbol] of VEGF (vascular endothelial growth factor) and Rk1, and incubated for 6 hours. After the incubation, the medium was washed with PBS solution and immunocytochemistry analysis was performed to determine the change of tight junction protein.

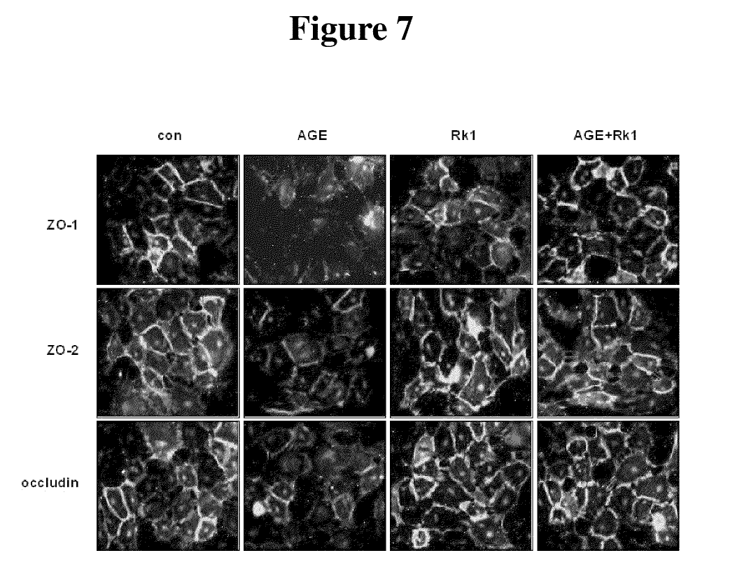

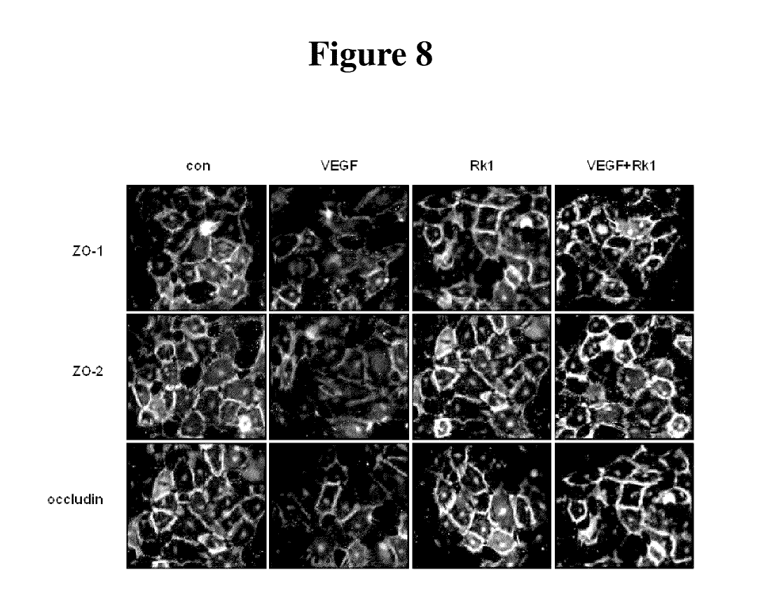

As shown in FIG. 7 and FIG. 8, Rk1 showed potent recovering effect on the decrease of tight junction protein, i.e., ZO-1, ZO-2 and occludin which was caused by VEGF and AGE (advanced glycation end product).

### 2-4. Immunocytochemistry (2)

Both of 100 ng of VEGF and 30 μg of Rk1 were injected into the retina of C57/BL6 mice (Orient, Korea). After 24 hours, retina was delivered from the mice and western blot analysis was performed.

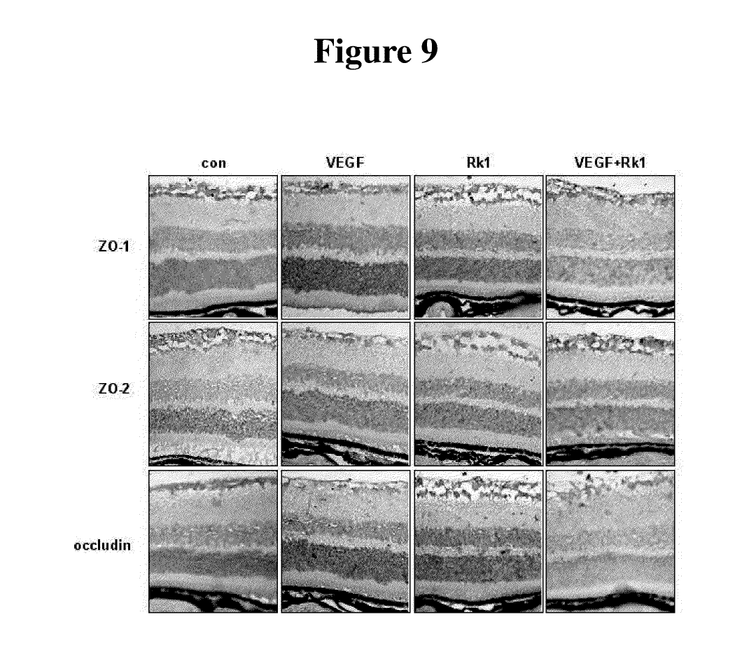

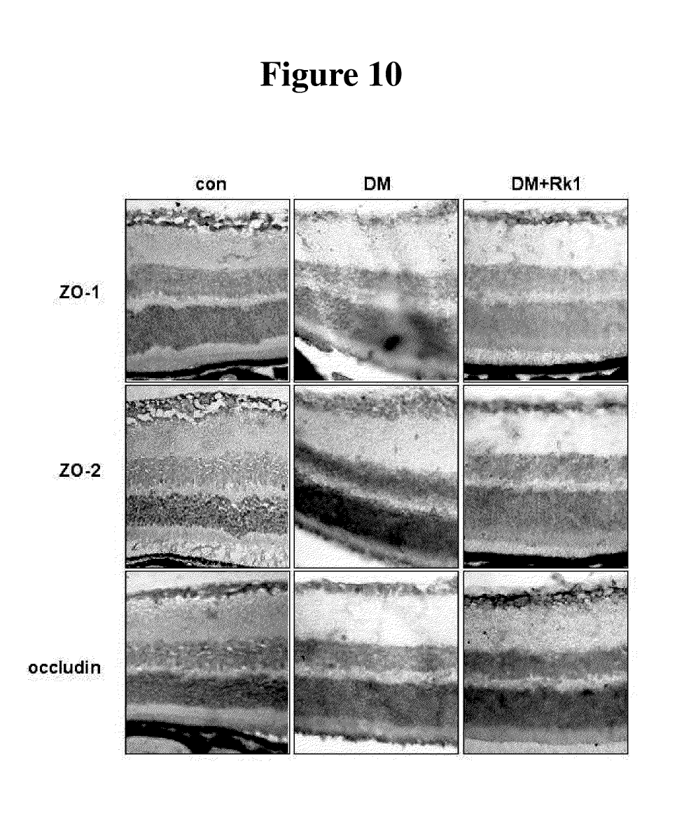

As shown in FIG. 9 and FIG. 10, Rk1 showed potent recovering effect on the decrease of tight junction protein, i.e., ZO-1, ZO-2 and occludin which was caused by VEGF and diabetes.

### 2-5. Angiography

Both of 100 ng of VEGF and 30 μg of Rk1 were injected into the retina of C57/BL6 mice (Orient, Korea). After 24 hours, the mice were anesthetized and FITC-dextran was injected into the heart to allow fluorescent material to flow in blood. After 30 minutes, the extracted retina from mice was performed to flat mounting and the change was observed with fluorescent microscope. In the only VEGF-injected mice, it has been confirmed that the fluorescent material was exuded from neighboring tissues around blood vessel caused by the increased permeability of retinal vessel however the exudation in VEGF was recovered by the treatment of Rk1.

Likewise, it has been also confirmed that the fluorescent material was exuded from neighboring tissues around blood vessel caused by the increased permeability of retinal vessel in diabetes induced mice however the exudation was recovered by the treatment of Rk1.

Hereinafter, the formulating methods and kinds of excipients will be described, but the present invention is not limited to them. The representative preparation examples were described as follows.

### Preparation of Injection

| | |
|---|---|
| Dried powder of Example 1 or ginsenoside Rg3 | 100 mg |
| Sodium metabisulfite | 3.0 mg |
| Methyl paraben | 0.8 mg |
| Propyl paraben | 0.1 mg |
| Distilled water for injection | optimum amount |

Injection preparation was prepared by dissolving active component, controlling pH to about 7.5 and then filling all the components in 2 m [symbol] ample and sterilizing by conventional injection preparation method.

### Preparation of Powder

| | |
|---|---|
| Dried powder mixture of ginsenoside Rg5 and Rk1 | 500 mg |
| Corn Starch | 100 mg |
| Lactose | 100 mg |
| Talc | 10 mg |

Powder preparation was prepared by mixing above components and filling sealed package.

### Preparation of Tablet

| | |
|---|---|
| Dried powder of Example 1 or ginsenoside Rg5 | 200 mg |
| Corn Starch | 100 mg |
| Lactose | 100 mg |
| Magnesium stearate | optimum amount |

Tablet preparation was prepared by mixing above components and entabletting.

### Preparation of Capsule

| | |
|---|---|
| Dried powder of Example 1 or ginsenoside Rk1 | 100 mg |
| Lactose | 50 mg |
| Corn starch | 50 mg |
| Talc | 2 mg |
| Magnesium stearate | optimum amount |

Tablet preparation was prepared by mixing above components and filling gelatin capsule by conventional gelatin preparation method.

### Preparation of Liquid

| | | |
|---|---|---|
| Dried powder of Example 1 or ginsenoside Rg3 | 1000 | mg |
| Sugar | 20 | g |
| Polysaccharide | 20 | g |
| Lemon flavor | 20 | g |

Liquid preparation was prepared by dissolving active component, and then filling all the components in 1000 m [symbol] ample and sterilizing by conventional liquid preparation method.

### Preparation of Health Food

| | |
|---|---|
| Dried powder of Example 1 or ginsenoside Rg5 | 1000 mg |
| Vitamin mixture | optimum amount |
| Vitamin A acetate | 70 μg |
| Vitamin E | 1.0 mg |
| Vitamin B₁ | 0.13 mg |
| Vitamin B₂ | 0.15 mg |
| Vitamin B6 | 0.5 mg |
| Vitamin B12 | 0.2 μg |
| Vitamin C | 10 mg |
| Biotin | 10 μg |
| Amide nicotinic acid | 1.7 mg |
| Folic acid | 50 μg |
| Calcium pantothenic acid | 0.5 mg |
| Mineral mixture | optimum amount |
| Ferrous sulfate | 1.75 mg |
| Zinc oxide | 0.82 mg |
| Magnesium carbonate | 25.3 mg |
| Monopotassium phosphate | 15 mg |
| Dicalcium phosphate | 55 mg |
| Potassium citrate | 90 mg |
| Calcium carbonate | 100 mg |
| Magnesium chloride | 24.8 mg |

The above mentioned vitamin and mineral mixture may be varied in many ways. Such variations are not to be regarded as a departure from the spirit and scope of the present invention.

### Preparation of Health Beverage

| | | |
|---|---|---|
| Dried powder of Example 1 or ginsenoside Rk1 | 1000 | mg |
| Citric acid | 1000 | mg |
| Oligosaccharide | 100 | g |
| Apricot concentration | 2 | g |
| Taurine | 1 | g |
| Distilled water | 900 | ml |

Health beverage preparation was prepared by dissolving active component, mixing, stirred at 85° C. for 1 hour, filtered and then filling all the components in 1000 m [symbol] ample and sterilizing by conventional health beverage preparation method.

The invention being thus described, it will be obvious that the same may be varied in many ways. Such variations are not to be regarded as a departure from the spirit and scope of the present invention, and all such modifications as would be obvious to one skilled in the art are intended to be included within the scope of the following claims.

### INDUSTRIAL APPLICABILITY

As described in the present invention, the extract of processed Panax genus plant and saponin compounds isolated therefrom show suppressive effect on the endothelial dysfunction and the injury of blood-retinal on HUVEC and retinal vascular endothelial cell. Therefore, it can be used as the therapeutics or functional health food for treating and preventing the diseases caused by vascular injury.

1. A use of an extract of processed Panax genus plants so as to make a ratio of ginsenoside (Rg3+Rg5+Rk1) to (Rb1+Rb2+Rc+Rd) of over 1.0 as an active ingredient for the preparation of the medicament employed for treating and preventing the diseases caused by vascular injury.

2. A use of an extract of processed ginseng obtained by the steps; treating Panax genus plants with heat at the temperature ranging from 70 to 150° C. for the period ranging from 2 to 6 hrs, as an active ingredient for the preparation of the medicament employed for treating and preventing the diseases caused by vascular injury.

3. The use according to claim 1, wherein said extract is extracted with the solvent selected from the group consisting of water, lower alcohols, acetone, ethyl acetate, chloroform, dichloromethane, and the mixtures thereof.

4. The use according to claim 1, wherein said Panax genus plants comprises the root, stem, petal, leaf or fruit of Panax ginseng, Panax quinquefolia, Panax notoginseng, Panax trifolia, Panax japonica, Panax pseudoginseng, Panax vietnamensis, Panax elegatior, Panax wangianus or Panax bipinratifidus.

5. A use of saponin compounds at least one or their combination selected from the group consisting of ginsenoside Rg3, Rg5 and Rk1, as an active ingredient for the preparation of the medicament employed for treating and preventing the diseases caused by vascular injury.

6. The use according to claim 1, wherein said diseases caused by vascular injury is ischemic diseases or diabetic complications.

7. (canceled)

8. (canceled)

9. (canceled)

10. (canceled)

11. A method for treating or preventing the diseases caused by vascular injury in mammals, wherein the method comprises administering a therapeutically effective amount of extract of processed ginseng or saponin compounds isolated therefrom as set forth in claim 1 into the mammal suffering with the diseases caused by vascular injury.

12. A pharmaceutical composition comprising an extract of processed ginseng and saponin compounds isolated therefrom as set forth in claim 1 as an active ingredient in an effective amount to treat and prevent the diseases caused by vascular injury.

13. A functional health food comprising an extract of processed ginseng or saponin compounds isolated therefrom as set forth in claim 1 together with a sitologically acceptable additive for the prevention and improvement of the disease caused by vascular injury.

14. The use according to claim 2, wherein said extract is extracted with the solvent selected from the group consisting of water, lower alcohols, acetone, ethyl acetate, chloroform, dichloromethane, and the mixtures thereof.

15. The use according to claim 2, wherein said Panax genus plants comprises the root, stem, petal, leaf or fruit of Panax ginseng, Panax quinquefolia, Panax notoginseng, Panax trifolia, Panax japonica, Panax pseudoginseng, Panax vietnamensis, Panax elegatior, Panax wangianus or Panax bipinratifidus.

16. The use according to claim 2, wherein said diseases caused by vascular injury is ischemic diseases or diabetic complications.

17. A method for treating or preventing the diseases caused by vascular injury in mammals, wherein the method comprises administering a therapeutically effective amount of extract of processed ginseng or saponin compounds isolated therefrom as set forth in claim 2 into the mammal suffering with the diseases caused by vascular injury.

18. A pharmaceutical composition comprising an extract of processed ginseng and saponin compounds isolated therefrom as set forth in claim 2 as an active ingredient in an effective amount to treat and prevent the diseases caused by vascular injury.

19. A functional health food comprising an extract of processed ginseng or saponin compounds isolated therefrom as set forth in claim 2 together with a sitologically acceptable additive for the prevention and improvement of the disease caused by vascular injury.

20. The use according to claim 5, wherein said diseases caused by vascular injury is ischemic diseases or diabetic complications.

21. A method for treating or preventing the diseases caused by vascular injury in mammals, wherein the method comprises administering a therapeutically effective amount of extract of processed ginseng or saponin compounds isolated therefrom as set forth in claim 5 into the mammal suffering with the diseases caused by vascular injury.

22. A pharmaceutical composition comprising an extract of processed ginseng and saponin compounds isolated therefrom as set forth in claim 5 as an active ingredient in an effective amount to treat and prevent the diseases caused by vascular injury.

23. A functional health food comprising an extract of processed ginseng or saponin compounds isolated therefrom as set forth in claim 5 together with a sitologically acceptable additive for the prevention and improvement of the disease caused by vascular injury.
